(19) United States
(12) Patent Application Publication
(10) Pub. No.: US 2023/0263785 A1
(43) Pub. Date: Aug. 24, 2023

(54) COMPANION DIAGNOSTIC FOR RTK INHIBITORS

(71) Applicant: The Regents of The University of Michigan, Ann Arbor, MI (US)

(72) Inventors: Paul Swiecicki, Ann Arbor, MI (US); J. Chad Brenner, Ann Arbor, MI (US)

(21) Appl. No.: 18/041,059

(22) PCT Filed: Aug. 10, 2021
(86) PCT No.: PCT/US2021/045373

§ 371 Date: Feb. 8, 2023

Related U.S. Application Data
(60) Provisional application No. 63/064,666, filed on Aug. 12, 2020.

(51) Int. Cl. A61K 31/44; C12Q 1/6886; A61K 45/06; C07K 16/22; A61K 31/47; A61K 31/404; A61K 31/506

(52) U.S. Cl. CPC A61K 31/44; C12Q 1/6886; A61K 45/06; C07K 16/22; A61K 31/47; A61K 31/404; A61K 31/506; C12Q 2600/106; C12Q 2600/156

(57) ABSTRACT

The disclosure provides methods for treating cancer with a receptor tyrosine kinase (RTK) inhibitor by first selecting a subject for treatment that has a mutation in one or more genes involved in the phosphoinositide 3-kinase (PI3K) signaling pathway, followed by administering the RTK inhibitor to the selected subject.

### CROSS REFERENCE TO RELATED APPLICATIONS

This application claims priority to U.S. Provisional Application No. 63/064,666, filed Aug. 12, 2020, which is hereby incorporated by reference in its entirety.

### FIELD

The present invention relates to methods of using mutations in phosphoinositide 3-kinase (PI3K) signaling pathway genes as a companion diagnostic for treating cancer patients with receptor tyrosine kinase inhibitors that target both a vascular endothelial growth factor receptor (VEGFR) and a fibroblast growth factor receptor (FGFR).

### BACKGROUND

New diagnostics are making it increasingly possible to personalize medical therapy by identifying patients who are more likely to benefit from a particular treatment, or who are at lower or higher risk for a particular side effect. While the concept of targeted treatments is not new, the emergence of novel biomarkers and diagnostics that can distinguish subsets of populations that respond differently to treatments is likely to result in a paradigm shift in the way patient treatment is managed. Rational drug development aligned with companion diagnostics for the identification of specific patient populations has the potential to achieve beneficial outcomes for patients and physicians, such as shorter development cycles and fewer treatment failures.

In cancer therapy, signaling pathways mediated by receptor tyrosine kinases (RTKs) have emerged as an important treatment target. Receptor tyrosine kinases (RTKs) are a subclass of tyrosine kinases that are involved in mediating cell-to-cell communication and controlling a wide range of complex biological functions, including, for example, cell growth, motility, differentiation, and metabolism. RTKs encompass a large superfamily of receptors for a wide array of growth factors, including epidermal growth factor (EGF), nerve growth factor (NGF), platelet-derived growth factor (PDGF), vascular endothelial growth factor (VEGF), fibroblast growth factor (FGF), insulin and the insulin-like growth factors (IGF), and the ephrins and angiopoietins. Dysregulation of RTK signaling leads to many human diseases, especially cancer. For example, VEGF dysregulation has been identified as a crucial process in the development of certain head and neck squamous cell carcinomas (HNSCC) (Strauss L, et al., Med Sci Monit, 11: BR280-92 (2005); and Hasina et al., Lab Invest, 88: 342-53 (2008)), and VEGF overexpression is associated with advanced disease and poor prognosis in various cancers (Smith et al., J Clin Oncol, 18: 2046-52 (2000); and Kyzas et al., Clin Cancer Res, 11: 1434-40 (2005). RTK inhibitors, such as VEGF signaling pathway (VSP) inhibitors, have been approved for several different malignancies, including, for example, axitinib, lenvatinib, regorafenib, and bevacizumab. Differential manifestations of response have been observed with certain RTK inhibitors, however, which have the potential of being incorrectly interpreted as progressive disease depending on the criteria employed. Patients may also acquire resistance to RTK inhibitors (Mok et al., N Engl J Med., 361: 947-57 (2009)). Acquired resistance can occur through either acquired genomic alterations (Kobayahsi et al., N Engl J Med., 352: 786-92 (2005)) or activation of critical signaling pathways (Blakely et al., Cell Rep., 11: 98-110 (2015)).

Thus, there remains a need for methods to more accurately identify cancer patients that will respond to therapy with RTK inhibitors.

### SUMMARY

The disclosure provides a method for treating a cancer in a subject, which method comprises: (a) determining the presence of a mutation in one or more genes involved in the phosphoinositide 3-kinase (PI3K) signaling pathway in a sample obtained from the subject; and (b) administering to the subject a receptor tyrosine kinase (RTK) inhibitor, wherein the RTK inhibitor is not axitinib, and whereby the cancer in the subject is treated.

The disclosure also provides a method for treating cancer in a subject, which method comprises. (a) determining the presence of a mutation in one or more genes involved in the phosphoinositide 3-kinase (PI3K) signaling pathway in a sample obtained from the subject; and (b) administering to the subject a receptor tyrosine kinase (RTK) inhibitor that targets a VEGF receptor (VEGFR) and a fibroblast growth factor receptor (FGFR), whereby the cancer in the subject is treated.

Also provided is a receptor tyrosine kinase (RTK) inhibitor for use in a method of treating a subject with cancer, wherein the method comprises: (a) determining whether a test sample from the subject comprises a mutation in one or more genes involved in the phosphoinositide 3-kinase (PI3K) signaling pathway; and (b) if the test sample from the subject comprises a mutation in one or more genes involved in the PI3K signaling pathway, administering to the subject an effective amount of the RTK inhibitor, wherein the RTK inhibitor is not axitinib.

### DETAILED DESCRIPTION OF THE INVENTION

### Definitions

To facilitate an understanding of the present technology, a number of terms and phrases are defined below. Additional definitions are set forth throughout the detailed description.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or a RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “variant” refers to the exhibition of qualities that have a pattern that deviates from what occurs in nature. In some embodiments, a variant may also be a mutant.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

The term “tumor,” as used herein, refers to an abnormal mass of tissue that results when cells divide more than they should or do not die when they should. In the context of the present disclosure, the term tumor may refer to tumor cells and tumor-associated stromal cells. Tumors may be benign and non-cancerous if they do not invade nearby tissue or spread to other parts of the organism. In contrast, the terms “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The terms “primary tumor” or “primary cancer” refer to an original, or first, tumor in the body. The term “metastasis,” as used herein, refers to the process by which cancer spreads from the location at which it first arose as a primary tumor to distant locations in the body. The terms “metastatic cancer” and “metastatic tumor” refer to the cancer or tumor resulting from the spread of a primary tumor. It will be appreciated that cancer cells of a primary tumor can metastasize through the blood or lymph systems.

An agent is “cytotoxic” and induces “cytotoxicity” if the agent kills or inhibits the growth of cells, particularly cancer cells. In some embodiments, for example, cytotoxicity includes preventing cancer cell division and growth, as well as reducing the size of a tumor or cancer. Cytotoxicity of tumor cells may be measured using any suitable cell viability assay known in the art, such as, for example, assays which measure cell lysis, cell membrane leakage, and apoptosis. For example, methods including but not limited to trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, Wis.), VIVAFIX™ 583/603 Cell Viability Assay (Bio-Rad, Hercules, Calif.); and CYTOTOX-FLUOR™ Cytotoxicity Assay (Promega, Madison, Wis.).

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition within the scope and meaning herein.

As used herein, the terms “treatment,” “treating,” and the like, refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

### Receptor Tyrosine Kinases

There are 58 known RTKs in humans (Manning et al., Science, 298: 1912-34 (2002); and Robinson et al., Oncogene, 19: 5548-57 (2000)). All RTKs contain an extracellular ligand binding domain, a single transmembrane helix, and an intracellular region that contains a juxtamembrane regulatory region, a tyrosine kinase domain (TKD) and a carboxyl (C-) terminal tail. RTKs are generally activated by receptor-specific ligands. Upon binding of ligands to extracellular regions of RTKs, the receptor is activated by ligand-induced receptor dimerization and/or oligomerization (Schlessinger, J., Cell, 103: 211-25 (2000)). For most RTKs, the resultant conformational changes enable trans-autophosphorylation of each TKD and release of cis-autoinhibition (Lemmon M A, Schlessinger J, Cell, 141: 1117-34 (2010)). This conformational change allows the TKD to assume an active conformation. Autophosphorylation of RTKs also recruits and activates a wide variety of downstream signaling proteins which contain Src homology-2 (SH2) or phosphotyrosine-binding (PTB) domains. SH2 and PTB domains bind to specific phosphotyrosine residues within the receptor and engage downstream mediators that promote critical cellular signaling pathways (Pawson et al., Trends Cell Biol., 11: 504-11 (2001); Du, Z., Lovly, C. M., Molecular Cancer, 17(58) (2018). doi: 10.1186/s12943-018-0782-4)). The 58 known RTKs can be divided into 20 subfamilies (Ségaliny et al., Journal of Bone Oncology, 4(1): 1-12 (2015), doi:10.1016/j.jbo.2015.01.001; Robinson et al., supra; and Blume-Jensen, P., Nature, 411: 355-365 (2001)). Such families include, but are not limited to, epidermal growth factor (EGF), nerve growth factor (NGF), platelet-derived growth factor (PDGF), vascular endothelial growth factor (VEGF), fibroblast growth factor (FGF), insulin and the insulin-like growth factors (IGF), and the ephrins (EPHR). As discussed above, dysregulation of RTK signaling leads to a variety of human diseases, such as cancers. Of the 20 RTK subfamilies, EGFR/ErbB (class I), the receptor for insulin (class II), the PDGF receptor (Class III), the FGF receptor (class IV), the VEGF (class V) receptor, and HGF (MET, Class VI) are strongly associated with oncological diseases.

Since RTKs play crucial roles in cancer development, degenerative diseases, and cardiovascular diseases, RTKs have become an attractive target for drug development. Several small-molecule inhibitors and monoclonal antibodies have been developed which target the extracellular domain or the catalytic domain of RTKs, thus inhibiting ligand binding and receptor oligomerization. Many of these inhibitors have been approved by the U.S. Food and Drug Administration (FDA) for treating a variety of cancers (e.g., colon cancer, renal cell carcinoma, lung cancer, and head and neck cancer), examples of which are listed in Table 1 below. As described in more detail herein, the inventive methods encompass administration of any suitable RTK inhibitor known in the art, with the exception of axitinib. Suitable RTK inhibitors are disclosed herein and are known in the art (see, e.g., Ferguson, F. M., Gray, N. S., Nature Reviews Drug Discovery, 17: 353-377(2018); Pottier et al., Cancers (Basel), 12(3): 731 (2020); doi:10.3390/cancers12030731; and Shawver et al., Cancer Cell, 1(2): 117-123 (2002). doi:10.1016/s1535-6108(02)00039-9). In some embodiments, the RTK inhibitor targets a VEGF receptor (VEGFR) and a fibroblast growth factor receptor (FGFR). The human VEGF family consists of five related glycoproteins: VEGFA (also known as VEGF), VEGFB, VEGFC, VEGFD, and PIGF (placental growth factor). The VEGF glycoproteins are secreted to form homodimers, which interact with a family of three receptor tyrosine kinases: VEGFR1 (VEGF receptor 1), VEGFR2, and VEGFR3. VEGFA and VEGFB bind to VEGFR1, VEGFA binds to VEGFR2, and VEGFC and VEGFD bind to both VEGFR2 and VEGFR3 (Ferrara N, Adamis A. P., Nat Rev Drug Discov., 15: 385-403 (2016) doi: 10.1038/nrd.2015.17; and Olsson et al., Nat Rev Mol Cell Biol., 7: 359-371 (2006) doi: 10.1038/nrm1911.). PIGF primarily interacts with VEGFR1. The VEGFRs are expressed on a wide variety of cell types. VEGFR1, also called Flt-1 (fms-like tyrosine kinase 1), is expressed on vascular endothelial cells, hematopoietic stem cells, monocytes, and macrophages. VEGFR2, also known as KDR (kinase insert domain) or Flk-1 (fetal liver kinase 1), is expressed on vascular and lymphatic endothelial cells; VEGFR3 (also known as Flt-4) expression is restricted to lymphatic endothelial cells (Olsson et al., supra). On ligand binding, VEGFRs transduce intracellular signals through a variety of mediators. In the case of VEGFR2, which is the best characterized, these include phosphotidylinositol-3 kinase (PI3K)/Akt, mitogen-activated kinases, the nonreceptor tyrosine kinase Src, as well as PLCγ (phospholipase C gamma)/PKC (protein kinase C), which promote angiogenesis, lymphangiogenesis, vascular permeability, and vascular homeostasis (Ferrara and Adamis, supra; and Olsson et al., supra). In some cases, VEGF proteins can use other receptors such as integrins (Koch et al., Biochem J., 437: 169-183 (2011)).

VEGF, together with its cognate receptor VEGFR2, is a major regulator of angiogenesis. Activation of the VEGF pathway has been implicated in a large number of disease processes, including cancer, autoimmunity, and retinopathy. As such, the development and use of agents that target and inhibit the VEGF signaling pathway has become an integral component of anticancer regimens for many tumor types. Indeed, the generation of a humanized neutralizing antibody to VEGF-A (bevacizumab/AVASTIN®) that benefits numerous human cancers has confirmed the merit of inhibiting VEGF signaling as a cancer treatment. Targeting other members of the VEGF family also is being explored for cancer therapy.

Several strategies have been used to inhibit VEGF signaling. Such strategies broadly include specific agents antagonizing the VEGFA/VEGFR2 axis or small molecule tyrosine kinase inhibitors (TKIs) exhibiting potent anti-VEGFR activity. Direct neutralization of VEGFA was the initial strategy that led to the development of bevacizumab. Soluble decoy receptors (receptor traps) may be used to sequester circulating VEGF to prevent downstream receptor activation; aflibercept functions in this role by mimicking the extracellular domains of VEGFR1 and VEGFR2, thus inhibiting the effects of VEGFA, VEGFB, and PIGF. Highly specific inhibitors of both the VEGF ligand (e.g., bevacizumab, VEGF-Trap, ranibizumab) as well as VEGF receptors (e.g., cediranib, pazopanib, sorafenib, sunitinib, vandetanib, telatinib, semaxanib, motesanib, vatalanib, and zactima) have either been approved or are under development for the treatment of a variety of cancers.

Fibroblast growth factors (FGFs) belong to a structurally related family of 22 molecules that interact with high-affinity tyrosine kinase FGF receptors (FGFRs) to regulate multiple fundamental pathways and cellular behaviors (Itoh N., Ornitz D. M., Trends in Genetics, 20: 563-9 (2004)). FGF signaling regulates events in embryonal development, including mesenchymal-epithelial signaling and the development of multiple organ systems (De Moerlooze et al., Development, 127: 483-92 (2000); Yamaguchi et al., Genes & Development, 8: 3032-44 (1994)). FGF signaling also regulates cell proliferation, differentiation, and survival as well as angiogenesis and wound healing (Baird et al., Recent Prog Horm Res., 42: 143-205 (1986)). In some situations, FGFs can act as a negative regulator of proliferation and positive regulator of differentiation. FGFRs are transmembrane tyrosine kinases that contain two or three extracellular immunoglobulin-like domains and an extracellular heparin-binding sequence (Lee et al., Science., 245: 57-60 (1989); Johnson, D., Williams, L., Adv Cancer Res., 60: 1-41 (1993); and McKeehan et al., Prog Nucleic Acid Res Mol Biol., 59: 135-76 (1998)). The two proximal immunoglobulin like extracellular domains bind the FGF ligand while the heparin-binding sequence binds a glycosaminoglycan moiety resulting in the formation of a complex containing two FGFs, two FGFRs, and the glycosaminoglycan moiety (Naski M. C., Ornitz D. M., Front Biosci., 3: D781-D94 (1998)). Only four FGFRs are known to exist, designated FGFR-1 through FGFR-4. Their specificity for various ligands is altered by processes that create multiple isoforms.

Aberrant FGF/FGFR signaling has been shown to play a role in cellular proliferation, resistance to cell death, increased motility and invasiveness, increased angiogenesis, enhanced metastasis, and resistance to chemotherapy (Kwabi-Addo et al., Edocr Relat Cancer, 11(4): 709-24 (2004)). Because aberrant FGF signaling can promote tumorigenesis by affecting major downstream biological processes, FGFs have been implicated in multiple tumor types, including prostate, astrocytoma, breast, lung, bladder, hepatocellular, and colon cancer (Turner N, Grose R., Nat Rev Cancer, 10: 116-29 (2010); and Lieu et al., Clin Cancer Res., 17(19): 6130-6139 (2011)). Evidence also suggests that up-regulation of FGF and FGFR may serve as a mechanism of resistance to anti-VEGF therapy (Casanovas et al., Cancer Cell, 8(4): 299-309 (2005); Allen et al., Clin Cancer Res., 17(16): 5299-310 (2011); Fernando et al., Clin Cancer Res., 14(5):1529-39 (2008); and Bello et al., Cancer Res., 71(4): 1396-405 (2011)). As such, FGFRs have been considered as promising drug targets for treating various cancers. Several FGFR inhibitors are in clinical trials or have been approved by the FDA, including, but not limited to, AZD4547 (Gavine et al., Cancer Res, 72(8): 2045-56 (2012)), erdafitinib (BALVERSA®), and TAS120 (Meric-Bernstam et al., Ann Oncol., 29 (suppl 5; abstr O-001) (2018)). FGFR inhibitors also are described in, e.g., Marseglia, et al., Expert Opinion on Therapeutic Patents, 29(12): 965-977 (2019), doi: 10.1080/13543776.2019.1688300).

### PI3K Status as Companion Diagnostic

Recent studies demonstrate that head and neck squamous cell carcinoma patients with mutations in genes involved in the phosphoinositide 3-kinase (PI3K) signaling pathway have been shown to respond better to treatment with an RTK inhibitor than HNSCC patients without such mutations. Thus, in some embodiments, mutations in PI3K pathway genes serve as biomarkers for response to treatment with an RTK inhibitor (e.g., an RTK inhibitor other than axitinib), and the methods described herein may be employed as a companion diagnostic to select cancer patients for therapy with any suitable RTK inhibitor. The U.S. Food and Drug Administration (FDA) defines a “companion diagnostic” as a medical device, often an in vitro device, which provides information that is essential for the safe and effective use of a corresponding drug or biological product. Furthermore, the FDA specifies four areas where a companion diagnostic assay could be essential: (i) to identify patients who are most likely to benefit from a particular therapeutic product; (ii) to identify patients likely to be at increased risk for serious side effects as a result of treatment with a particular therapeutic product; (iii) to monitor response to treatment with a particular therapeutic product for the purpose of adjusting treatment to achieve improved safety or effectiveness, and (iv) to identify patients in the population for whom the therapeutic product has been adequately studied, and found safe and effective, i.e., there is insufficient information about the safety and effectiveness of the therapeutic product in any other population (US FDA. Guidance for Industry and Food and Drug Administration Staff. In Vitro Companion Diagnostic Devices. Aug. 6, 2014; Jørgensen, J. T. and M. Hersom, Ann Transl Med., 4(24): 482 (2016); and Agarwal et al., Pharmgenomics Pers Med., 8: 99-110 (2015). Currently, the term “companion diagnostic” is understood in the art as also encompassing a diagnostic test or biomarker used in a specific context that provides biological and/or clinical information that enables better decision making about the development and use of a potential drug therapy (Austin, M J F. Companion Diagnostics: Reality Check. Cambridge Healthtech Institute (CHI) Next Generation Dx Summit. Pre-Conference Symposium, Aug. 9, 2009. Washington D.C, USA; and Frueh, F. Reality Check on Companion Diagnostics. Cambridge Healthtech Institute (CHI) Next Generation Dx Summit. Pre-Conference Symposium, Aug. 9, 2009. Washington D.C, USA).

The disclosure provides a method for treating a cancer in a subject, which comprises determining the presence of a mutation in one or more genes involved in the phosphoinositide 3-kinase (PI3K) signaling pathway in a sample obtained from the subject. The PI3K (also referred to in the art as “PI3K/AKT” and “(PI3K)/AKT/mammalian target of rapamycin (mTOR)”) signaling pathway is a key regulator of normal cellular processes involved in cell growth, proliferation, metabolism, motility, survival, and apoptosis (Katso et al., Annu Rev Cell Dev Biol., 17: 615-75 (2001); and Engelman et al., Nat Rev Genet., 7: 606-19 (2006)). Aberrant activation of the PI3K pathway promotes the survival and proliferation of tumor cells in many human cancers, as well as resistance to anticancer therapies (Porta et al., Front Oncol., 4: 64 (2014); Huang et al., J Formos Med Assoc., 108: 180-194 (2009); and Martini et al., Ann Med., 46: 372-83 (2014)). PI3K, AKT, a serine/threonine protein kinase also known as protein kinase B (PKB), and mTOR are three major proteins in the pathway. These proteins are typically activated by upstream signaling of tyrosine kinases and other receptor molecules such as hormones and mitogenic factors (Ruggero et al., Oncogene, 24: 7426-7434 (2005)).

The inventive method encompasses determining the presence of at least one mutation in any gene encoding a protein that is involved in the PI3K signaling pathway. Exemplary genes include, but are not limited to, XIAP/BIRC4 (X-linked inhibitor of apoptosis; NM_001167.2); AKT1 (v-akt murine thymoma viral oncogene homolog 1; NM_005163); TWIST1 (Twist homolog 1 (Drosophila); NM_000474.3); BAD (BCL2-associated agonist of cell death NM_004322.2), CDKN1A/p21 (Cyclin-dependent kinase inhibitor 1A (p21, Cip1); NM_000389.2); ABL1 (v-abl Abelson murine leukemia viral oncogene homolog 1; NM_005157.3); CDH1 (Cadherin 1, type 1, E-cadherin; NM_004360.3); TP53 (Tumor protein p53; NM_000546); CASP3 (Caspase 3, apoptosis-related cysteine peptidase; NM_004346.2); PAKI (p21/Cdc42/Rac1-activated kinase 1; NM_002576.4); GAPDH (Glyceraldehyde-3-phosphate dehydrogenase; NM_002046.3); PIK3CA (Phosphoinositide-3-kinase, catalytic, a-polypeptide; NM_006218.2); FAS (TNF receptor superfamily, member 6; NM_000043.3); AKT2 (v-akt murine thymoma viral oncogene homolog 2; NM_001626.3); FRAP1/mTOR (FK506 binding protein 12-rapamycin associated protein 1; NM_004958.3); FOXO1A (Forkhead box 01; NM_002015.3); PTK2 (FAK) (PTK2 protein tyrosine kinase 2; NM_005607.3); CASP9 (Caspase 9, apoptosis-related cysteine peptidase; NM_001229.2); PTEN (Phosphatase and tensin homolog; NM_000314.4); CCND1 (Cyclin D1; NM_053056.2); NFKB1 (Nuclear factor k-light polypeptide gene enhancer B-cells 1; NM_003998.2); GSK3B (Glycogen synthase kinase 3-b; NM_002093.2); MDM2 (Mdm2 p53 binding protein homolog (mouse); NM_002392.2); and CDKN1B (Cyclin-dependent kinase inhibitor 1B (p27, Kip1); NM_004064.3) (see, e.g., Catasus et al., Modern Pathology, 23: 694-702 (2010); Vivanco I, Sawyers C L., Nat Rev Cancer, 2: 489-501 (2002); Cully et al., Nat Rev Cancer, 6: 184-192 (2006); Bader et al., Nat Rev Cancer, 5: 921-929 (2005); and Samuels Y, Ericson K., Curr Opin Oncol, 18: 77-82 (2006)). In some embodiments, the method involves determining the presence of at least one mutation in one or more genes selected from PTEN, PIK3CA, and AKT.

The term “mutation,” as used herein, encompasses any structural change made to a wild-type nucleic acid sequence. The one or more PI3K pathway genes may have any type of mutation, and the mutation may or may not result in a protein with altered function. In some embodiments, however, the one or more mutations impairs the function of protein encoded by the PI3K pathway gene. For example, the mutation may be a missense mutation, a nonsense mutation, deletion or insertion of one or more nucleotides, duplication, amplification, a frameshift mutation, repeat expansion, and/or other modifications that affect the structural integrity or nucleotide sequence. A “missense mutation” is a change in one DNA base pair that results in the substitution of one amino acid for another in the encoded protein. A “nonsense mutation” is a change in one DNA base pair that converts a sense codon to a chain-terminating codon, resulting in the translation of an abnormally short polypeptide generally with altered functionality. A “duplication” comprises a piece of DNA that is abnormally copied one or more times. “Amplification” is a mutation that increases the copy number of a specific DNA segment in a cell. A “frameshift mutation” occurs when the addition or loss of DNA bases changes a gene's reading frame. Insertions, deletions, and duplications can all induce frameshift mutations. “Repeat expansion” refers to a mutation that increases the number of times that a short (e.g., 3 or 4 base pairs) DNA sequence present in the gene is repeated.

The presence of a mutation in one or more genes involved in the PI3K signaling pathway may be determined using any suitable method, a variety of which are known in the art. Such methods include, for example, restriction fragment length polymorphism (RFLP) analysis, Sanger sequencing, high-throughput sequencing (also referred to as “next generation sequencing”), tracking of indels by decomposition (TIDE) software, T7 endonuclease 1 (T7E1) assay, PCR based methods (e.g., RT-PCR, real-time or quantitative PCR, multiplex PCR, and nested PCR), multiplex ligation-dependent probe amplification (MLPA), denaturing gradient gel electrophoresis (DGGE), single strand conformational polymorphism (SSCP), chemical cleavage of mismatch (CCM), protein truncation test (PTT), and oligonucleotide ligation assay (OLA) (see, e.g., Mahdieh, N. and B. Rabbani, Iran J. Pediatr., 23(4): 375-388 (2013); Al-Haggar, M., Gene Technology 2: e104 (2013). doi: 10.4172/2329-6682.1000e104; and Frayling et al., PCR-Based Methods for Mutation Detection. In: Coleman W. B., Tsongalis G. J. (eds) Molecular Diagnostics. Humana Press (2006)).

### Sample

The terms “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. In some embodiments, the biological sample may comprise tumor tissue, suspected tumor tissue, or lymph node tissue. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. It will be appreciated that obtaining a biological sample from a subject may comprise extracting the biological sample directly from the subject or receiving the biological sample from a third party.

### Treatment of Cancer

The disclosure provides a method of treating cancer. Ideally, administration of an RTK inhibitor as described herein inhibits the growth of cancer cells from a primary tumor or cancer or a metastatic tumor or cancer. In some embodiments, the method induces cytotoxicity in tumor cells or cancer cells.

A cancer or tumor may arise in any organ or tissue. For example, the cancer or tumor may be a carcinoma (cancer arising from epithelial cells), a sarcoma (cancer arising from bone and soft tissues), a lymphoma (cancer arising from lymphocytes), a melanoma, or brain and spinal cord tumors. The tumor or cancer cells can arise in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). More particularly, tumors or cancers of the digestive system can arise in the esophagus, stomach, small intestine, colon, rectum, anus, liver, gall bladder, and pancreas. Cancers or tumors of the respiratory system can arise in the larynx, lung, and bronchus and include, for example, non-small cell lung carcinoma. Cancers or tumors of the reproductive system can affect the uterine cervix, uterine corpus, ovaries, vulva, vagina, prostate, testis, and penis. Cancers of the urinary system can arise in the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, etc.). The cancer may be a primary tumor, or alternatively, or a metastatic tumor. In some embodiments, the cancer is a head and neck cancer, such as a squamous cell head and neck carcinoma (HNSCC) or unresectable recurrent or metastatic head and neck squamous cell carcinoma (R/M HNSCC), colorectal cancer (CRC), ovarian cancer, breast cancer, non-small-cell lung cancer (NSCLC), renal cell cancer, glioblastoma, hepatocellular carcinoma (HCC), or chronic lymphocytic leukemia (CLL).

Targeted therapy has demonstrated promise in pre-clinical studies in HNSCC. Alterations in PI3KCA, CDKN2A, and EGFR suggest head and neck cancer is a candidate for the development of targeted therapeutics. RTK inhibitors offer the benefit of targeting numerous pathways (i.e., VEGFR, FGFR, EGFR, PDGFR) and isoforms simultaneously. For example, evidence suggests that VEGF inhibition is immunomodulatory via numerous mechanisms, including production of IFNγ, reversal of the immunosuppressive microenvironment, and augmented activity of CD8+ T cells via hypoxia-inducible factor-la secondary to tumor hypoxia¹⁹⁻²¹. As VEGFR inhibition may prime the immune system for response to immunotherapy, sequential use may be a modality to decrease toxicities yet still gain therapeutic synergy.

Once a subject is determined to have at least one mutation in one or more genes involved in the PI3K signaling pathway, the method comprises administering an RTK inhibitor to the subject, whereby the cancer in the subject is treated. In some embodiments, the RTK inhibitor is not axitinib. Thus, the disclosure also provides a receptor tyrosine kinase (RTK) inhibitor for use in a method of treating a subject with cancer, wherein the method comprises: (a) determining whether a test sample from the subject comprises a mutation in one or more genes involved in the PI3K signaling pathway: and (b) if the test sample from the subject comprises a mutation in one or more genes involved in the PI3K signaling pathway, administering to the subject an effective amount of the RTK inhibitor, wherein the RTK inhibitor is not axitinib. The term “RTK inhibitor,” as used herein, refers to any substance, compound, or agent that interferes with the expression and/or biological activity or function of one or more receptor tyrosine kinases, such as those described herein. The degree of inhibition may be partially complete (e.g., 10% or more, 25% or more, 50% or more, or 75% or more), substantially complete (e.g., 85% or more, 90% or more, or 95% or more), or fully complete (e.g., 98% or more, or 99% or more). Inhibition of RTK-mediated signaling as disclosed herein may involve interfering with or inhibiting the biological activity of a receptor tyrosine kinase (e.g., VEGFR and/or FGFR) and/or the expression of a receptor tyrosine kinase.

Any suitable RTK inhibitor or combinations of RTK inhibitors may be used in the context of the disclosed method. As discussed above, several RTK inhibitors have been approved for use to treat a variety of different cancers, or are currently under investigation. In some embodiments, the RTK inhibitor may be an agent that targets (i.e., inhibits the activity of) both a VEGF receptor (VEGFR) and a fibroblast growth factor receptor (FGFR). The RTK inhibitor may inhibit any VEGFR (e.g., VEGFR1, VEGFR2, VEGFR3, or combinations thereof) and any FGFR (e.g., FGFR1, FGFR2, FGFR3, FGFR4, or combinations thereof). Exemplary RTK inhibitors that may be used in the disclosed methods are listed in Table 1, but the disclosure is not limited to these particular inhibitors. In some embodiments, the RTK inhibitor is lenvatinib (LENVIMA®), which is small molecule inhibitor of multiple RTKs, including VEGFRs and FGFRs.

TABLE 1
Examples of RTK Inhibitors

| | Inhibitor | |
|---|---|---|
| RTK Inhibitor | Description | FDA-Approved Indication |
| Bevacizumab | Humanized | 1^{st} line metastatic colorectal |
| (AVASTIN ®); | anti-VEGF | cancer (mCRC) with |
| Genentech) | mAb | intravenous 5′-FU- |
| | | based chemotherapy; |
| | | 2^{nd} line mCRC with |
| | | intravenous 5′-FU-based |
| | | chemotherapy; |
| | | 1^{st} line NSCLC with |
| | | carboplatin and paclitaxel; |
| | | 1^{st} line renal with |
| | | interferon alfa; |
| | | 2^{nd} line GBM |
| | | as monotherapy; |
| | | 1^{st} line MBC with paclitaxel; |
| | | currently being reconsidered |
| Regorafenib | Small molecule | Last line mCRC; |
| (STIVARGA ®; | tyrosine kinase | Last line locally advanced, |
| Bayer) | inhibitor (TKI): | unresectable or metastatic |
| | BRAF, VEGFR-1, | gastrointestinal stromal |
| | -2, -3, KIT, | tumor (GIST); |
| | TIE-2, PDGFR-β, | 2^{nd} line hepatocellular |
| | FGFR-1, RET | carcinoma (HCC) |
| | and RAF-1 | |
| Lenvatinib | Small molecule | locally recurrent |
| (LENVIMA ®; | TKI: | or metastatic, |
| Eisai Inc.) | VEGFR1, | progressive, radioactive |
| | VEGFR2 and | iodine-refractory |
| | VEGFR3, FGFRs | differentiated |
| | | thyroid cancer; |
| | | In combination |
| | | with everolimus, |
| | | for advanced renal cell |
| | | carcinoma (RCC) |
| | | following one |
| | | prior antiangiogenic therapy; |
| | | 1^{ST} line unresectable HCC; |
| | | in combination with |
| | | pembrolizumab, for |
| | | advanced endometrial |
| | | carcinoma |
| | | that is not microsatellite |
| | | instability-high (MSI-H) or |
| | | mismatch repair deficient |
| | | (dMMR) |
| Sunitinib | Small molecule TKI: | 1^{st} line renal as monotherapy; |
| (SUTENT ®; | VEGFRs, PDGRs, | 2^{nd} line GIST as |
| Pfizer) | c-kit, Flt3, Ret | monotherapy |
| Sorafenib | Small molecule TKI: | 1^{st} line renal as monotherapy; |
| (NEXAVAR ®; | VEGFRs, PDGRs, | 1^{st} line HCC as monotherapy |
| Onyx/Bayer) | c-kit, Ret, Raf | |
| Pazopanib | Small molecule TKI: | 1^{st} line renal as monotherapy |
| (VOTRIENT ®); | VEGFRs, PDGRs, | |
| GlaxoSmith- | c-kit | |
| Kline) | | |
| Brivanib | Small molecule TKI: | Under investigation |
| alaninate | VEGFs, PDGFRs, | |
| (Bristol-Myers | FGFRs | |
| Squibb) | | |
| Cediranib | Small molecule TKI: | Under investigation |
| (RECENTIN ®); | VEGFRs, c-kit, | |
| AstraZeneca) | PDGFRs | |
| Vandetanib | Small molecule TKI: | symptomatic or progressive |
| (CAPRELSA ®); | VEGFRs, PDGFRS, | medullary thyroid cancer in |
| Sanofi) | EGFR, Ret | patients with unresectable |
| | | locally advanced or |
| | | metastatic disease |
| Linifinib | Small molecule TKI: | Under investigation |
| (Abbott) | VEGRs, PDGFRs | |
| Aflibercept | Recombinant | in combination |
| (ZALTRAP ®); | VEGFR | with fluorouracil, |
| Regeneron/ | fusion protein | leucovorin, irinotecan |
| Sanofi- | that binds | (FOLFIRI ®) for mCRC |
| Aventis) | VEGF A and | resistant to or progressed |
| | B, PIGF | following an oxaliplatin- |
| | | containing regimen |

Any suitable dose of the RTK inhibitor may be administered to the subject, so long as the RTK inhibitor is efficiently delivered to target cancer cells such that cancer cell growth is inhibited. To this end, the inventive method comprises administering a “therapeutically effective amount” of the RTK inhibitor. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the RTK inhibitor to elicit a desired response in the individual. For example, a therapeutically effective amount of a RTK inhibitor is an amount which is cytotoxic to cancer cells, such that the cancer or tumor is eliminated.

The RTK inhibitor may be formulated for administration to a mammal, particularly a human, using standard administration techniques and routes. Suitable administration routes include, but are not limited to, oral, intravenous, intraperitoneal, subcutaneous, subcutaneous, intramuscular, or parenteral administration. The term “parenteral,” as used herein, includes intravenous, intramuscular, subcutaneous, rectal, vaginal, and intraperitoneal administration. In other embodiments, the formulation may be administered to a mammal using systemic delivery by intravenous, intramuscular, intraperitoneal, or subcutaneous injection.

In some embodiments, the subject has received at least one cancer treatment prior to the administration of the dose of the RTK inhibitor. The subject may have previously received any cancer treatment known in the art, such as, for example, surgery, chemotherapy, radiation therapy, or cancer immunotherapy, hormone therapy, and/or stem cell transplantation. Chemotherapeutic agents include, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, and the like. In other embodiments, the subject has been treated with an immune checkpoint inhibitor (discussed further below) prior to the administration of the dose of RTK inhibitor. For example, the subject may have received treatment with a PD-1 inhibitor prior to administration of the dose of RTK inhibitor.

In some embodiments, the disclosed method promotes inhibition of cancer cell proliferation, the eradication of cancer cells, and/or a reduction in the size of at least one cancer or tumor such that the cancer or tumor is treated in a mammal (e.g., a human). By “treatment of cancer” is meant alleviation of a cancer in whole or in part. In one embodiment, the disclosed method reduces the size of a cancer or tumor by at least about 20% (e.g., cancer about 25%, about 30%, about 35%, about 40%, about 45%, about 50%, about 55%, about 60%, about 65%, about 70%, about 75%, about 80%, about 85%, about 90%, or about 95%). Ideally, the cancer or tumor is completely eliminated.

In some embodiments, the disclosed method may further comprise administering a cancer immunotherapeutic to the subject simultaneously with or subsequently to administration of the dose of the RTK inhibitor. A “cancer immunotherapeutic” is any agent, substance, compound, or method used to treat cancer that involves or uses components of a patient's immune system. In some embodiments, cancer immunotherapeutics may include antibodies that bind to, and inhibit the function of, proteins expressed by cancer cells. Other cancer immunotherapies include vaccines and T cell infusions. Thus, the cancer immunotherapeutic used herein may include, for example, immune checkpoint inhibitors, monoclonal antibodies, cancer vaccines, immune system modulators, and/or T-cell transfer therapy. Cancer immunotherapy is further described in, e.g., Finck et al., Nat Commun, 11: 3325 (2020). doi.org/10.1038/s41467-020-17140-5; and Karp et al. (eds.), Handhook of Targeted Cancer Therapy and Immunotherapy, Second Edition, Lippincott, Williams & Wilkins, 408 pp. (2018)).

In some embodiments, the method further comprises administering to the subject an immune checkpoint regulator. Immune checkpoints are molecules on immune cells that must be activated or inhibited to stimulate immune system activity. Tumors can use such checkpoints to evade attacks by the immune system. The immune checkpoint regulator may be an antagonist of an inhibitory signal of an immune cell, also referred to as a “checkpoint inhibitor,” which blocks inhibitory checkpoints (i.e., molecules that normally inhibit immune responses). For example, the immune checkpoint regulator may be an antagonist of A2AR, BTLA, B7-H3, B7-H4, CTLA4, GAL9, IDO, KIR, LAG3, PD-1, TDO, TIGIT, TIM3 and/or VISTA. Checkpoint inhibitor therapy therefore can block inhibitory checkpoints, restoring immune system function. Currently approved checkpoint inhibitors target the molecules CTLA4, PD-1, and PD-L1, and include ipilimumab (YERVOY®), nivolumab (OPDIVO®), pembrolizumab (KEYTRUDA®), atezolizumab (TECENTRIQ®), avelumab (BAVENCIO®), and durvalumab (IMFINZI®). Any suitable checkpoint inhibitor, such as those described in, e.g., Kyi, C. and M. A. Postow, Immunotherapy, 8(7): 821-37 (2016); Collin, M., Expert Opin Ther Pat., 26(5): 555-64 (2016); Pardoll, D. M., Nat Rev, Cancer, 12(4): 252-6 (2012); and Gubin et al., Nature, 5/5(7528): 577-81 (2014)) may be used in combination with the disclosed method. In other embodiments, the immune checkpoint regulator may be an agonist of an immune cell stimulatory receptor, such as an agonist of BAFFR, BCMA, CD27, CD28, CD40, CD122, CD137, CD226, CRTAM, GITR, HVEM, ICOS, DR3, LTBR, TACI and/or OX40.

### REFERENCES

All references, including publications, patent applications, and patents, cited herein are hereby incorporated by reference to the same extent as if each reference were individually and specifically indicated to be incorporated by reference and were set forth in its entirety herein.
1. Chaturvedi A K, Engels E A, Pfeiffer R M, et al: Human papillomavirus and rising oropharyngeal cancer incidence in the United States. J Clin Oncol 29:4294-301, 2011
2. Siegel R L, Miller K D, Jemal A: Cancer statistics, 2020. CA Cancer J Clin 70:7-30, 2020
3. Mandal R, Senbabaoglu Y, Desrichard A, et al: The head and neck cancer immune landscape and its immunotherapeutic implications. JCI Insight 1:e89829, 2016
4. Burtness B, Harrington K J, Greil R, et al: Pembrolizumab alone or with chemotherapy versus cetuximab with chemotherapy for recurrent or metastatic squamous cell carcinoma of the head and neck (KEYNOTE-048): a randomised, open-label, phase 3 study. Lancet 394:1915-1928, 2019
5. Cancer Genome Atlas N: Comprehensive genomic characterization of head and neck squamous cell carcinomas. Nature 517:576-82, 2015
6. Strauss L, Volland D, Kunkel M, et al: Dual role of VEGF family members in the pathogenesis of head and neck cancer (HNSCC): possible link between angiogenesis and immune tolerance. Med Sci Monit 11:BR280-92, 2005
7. Hasina R, Whipple M E, Martin L E, et al: Angiogenic heterogeneity in head and neck squamous cell carcinoma: biological and therapeutic implications. Lab Invest 88:342-53, 2008
8. Smith B D, Smith G L, Carter D, et al: Prognostic significance of vascular endothelial growth factor protein levels in oral and oropharyngeal squamous cell carcinoma. J Clin Oncol 18:2046-52, 2000
9. Kyzas P A, Cunha I W, Ioannidis J P: Prognostic significance of vascular endothelial growth factor immunohistochemical expression in head and neck squamous cell carcinoma: a meta-analysis. Clin Cancer Res 11:1434-40, 2005
10. Ongkeko W M, Altuna X, Weisman R A, et al: Expression of protein tyrosine kinases in head and neck squamous cell carcinomas. Am J Clin Pathol 124:71-6, 2005
11. Bran B, Bran G, Hormann K, et al: The platelet-derived growth factor receptor as a target for vascular endothelial growth factor-mediated anti-angiogenetic therapy in head and neck cancer. Int J Oncol 34:255-61, 2009
12. Swiecicki P L, Zhao L, Belile E, et al: A phase II study evaluating axitinib in patients with unresectable, recurrent or metastatic head and neck cancer. Invest New Drugs 33:1248-56, 2015
13. Choi H, Charnsangavej C, Faria S C, et al: Correlation of computed tomography and positron emission tomography in patients with metastatic gastrointestinal stromal tumor treated at a single institution with imatinib mesylate: proposal of new computed tomography response criteria. J Clin Oncol 25:1753-9, 2007
14. Herbst R S, Arquette M, Shin D M, et al: Phase II multicenter study of the epidermal growth factor receptor antibody cetuximab and cisplatin for recurrent and refractory squamous cell carcinoma of the head and neck. J Clin Oncol 23:5578-87, 2005
15. Motzer R J, Escudier B, Tomczak P, et al: Axitinib versus sorafenib as second-line treatment for advanced renal cell carcinoma: overall survival analysis and updated results from a randomised phase 3 trial. Lancet Oncol 14:552-62, 2013
16. Hutson T E, Lesovoy V, Al-Shukri S, et al: Axitinib versus sorafenib as first-line therapy in patients with metastatic renal-cell carcinoma: a randomised open-label phase 3 trial. Lancet Oncol 14:1287-94, 2013
17. Vermorken J B, Trigo J, Hitt R, et al: Open-label, uncontrolled, multicenter phase II study to evaluate the efficacy and toxicity of cetuximab as a single agent in patients with recurrent and/or metastatic squamous cell carcinoma of the head and neck who failed to respond to platinum-based therapy. J Clin Oncol 25:2171-7, 2007
18. Vermorken J B, Mesia R, Rivera F, et al: Platinum-based chemotherapy plus cetuximab in head and neck cancer. N Engl J Med 359:1116-27, 2008
19. Terme M, Pernot S, Marcheteau E, et al: VEGFA-VEGFR pathway blockade inhibits tumor-induced regulatory T-cell proliferation in colorectal cancer. Cancer Res 73:539-49, 2013
20. Doedens A L, Phan A T, Stradner M H, et al: Hypoxia-inducible factors enhance the effector responses of CD8(+) T cells to persistent antigen. Nat Immunol 14:1173-82, 2013
21. de Almeida P E, Mak J, Hernandez G, et al: Anti-VEGF Treatment Enhances CD8(+) T-cell Antitumor Activity by Amplifying Hypoxia. Cancer Immunol Res, 2020
22. Seiwert T Y, Burtness B, Mehra R, et al: Safety and clinical activity of pembrolizumab for treatment of recurrent or metastatic squamous cell carcinoma of the head and neck (KEYNOTE-012): an open-label, multicentre, phase 1b trial. Lancet Oncol 17:956-965, 2016
23. Ferris R L, Blumenschein G, Jr., Fayette J, et al: Nivolumab for Recurrent Squamous-Cell Carcinoma of the Head and Neck. N Engl J Med 375:1856-1867, 2016
24. Taylor M H R D, Brose M S, et al.: A phase 1b/2 trial of lenvatinib plus pembrolizumab in patients with squamous cell carcinoma of the head and neck. J Clin Oncol 36:6016, 2018
25. Wolchok J D, Hoos A, O'Day S, et al: Guidelines for the evaluation of immune therapy activity in solid tumors: immune-related response criteria. Clin Cancer Res 15:7412-20, 2009
26. Karar J, Maity A: PI3K/AKT/mTOR Pathway in Angiogenesis. Front Mol Neurosci 4:51, 2011
27. Manyam B V, Garsa A A, Chin R I, et al: A multi-institutional comparison of outcomes of immunosuppressed and immunocompetent patients treated with surgery and radiation therapy for cutaneous squamous cell carcinoma of the head and neck. Cancer 123:2054-2060, 2017
28. Venables Z C, Autier P, Nijsten T, et al: Nationwide Incidence of Metastatic Cutaneous Squamous Cell Carcinoma in England. JAMA Dermatol 155:298-306, 2019

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating a cancer in a subject, which method comprises:
(a) determining the presence of a mutation in one or more genes involved in the phosphoinositide 3-kinase (PI3K) signaling pathway in a sample obtained from the subject; and
(b) administering to the subject a receptor tyrosine kinase (RTK) inhibitor, wherein the RTK inhibitor is not axitinib, and whereby the cancer in the subject is treated.

2. A method for treating cancer in a subject, which method comprises:
(a) determining the presence of a mutation in one or more genes involved in the phosphoinositide 3-kinase (PI3K) signaling pathway in a sample obtained from the subject; and
(b) administering to the subject a receptor tyrosine kinase (RTK) inhibitor that targets a VEGF receptor (VEGFR) and a fibroblast growth factor receptor (FGFR), whereby the cancer in the subject is treated.

3. The method of claim 1 or claim 2, wherein the cancer is unresectable recurrent or metastatic head and neck squamous cell carcinoma (R/M HNSCC), ovarian cancer, breast cancer, renal cell cancer (RCC), colorectal cancer (CRC), non-small-cell lung cancer (NSCLC), hepatocellular carcinoma (HCC), or chronic lymphocytic leukemia (CLL).

4. The method of any one of claims 1-3, wherein the one or more genes involved in the PI3K signaling pathway are selected from PTEN, PIK3CA, and AKT.

5. The method of any one of claims 1-4, wherein the RTK inhibitor is selected from bevacizumab, lenvatinib, regorafenib, sunitinib, sorafenib, and pazopanib.

6. The method of any one of claims 1-5, wherein the sample comprises blood, tumor tissue or suspected tumor tissue, lymph node tissue, urine, or saliva.

7. The method of any one of claims 1-6, which further comprises administering a cancer immunotherapeutic to the subject simultaneously with or subsequently to administration of the RTK inhibitor.

8. The method of claim 7, wherein the cancer immunotherapeutic is selected from immune checkpoint inhibitors, monoclonal antibodies, cancer vaccines, immune system modulators, and T-cell transfer therapy.

9. The method of claim 8, wherein the cancer immunotherapeutic is an immune checkpoint inhibitor.

10. The method of claim 9, wherein the immune checkpoint inhibitor is a PD-1 inhibitor.

11. The method of any one of claims 1-10, wherein the subject is a human.

12. A receptor tyrosine kinase inhibitor for use in a method of treating a subject with cancer, wherein the method comprises:
(a) determining whether a test sample from the subject comprises a mutation in one or more genes involved in the phosphoinositide 3-kinase (PI3K) signaling pathway; and
(b) if the test sample from the subject comprises a mutation in one or more genes involved in the PI3K signaling pathway, administering to the subject an effective amount of the RTK inhibitor, wherein the RTK inhibitor is not axitinib.
